(19) United States
(12) Patent Application Publication
(10) Pub. No.: US 2017/0014265 A1
(43) Pub. Date: Jan. 19, 2017

(54) Microwaveable Compression Gloves

(71) Applicants: Mary Ann Russ, Mobile, AL (US); Samuel Hardie Russ, Mobile, AL (US)

(72) Inventors: Mary Ann Russ, Mobile, AL (US); Samuel Hardie Russ, Mobile, AL (US)

(21) Appl. No.: 15/211,500

(22) Filed: Jul. 15, 2016

Related U.S. Application Data
(60) Provisional application No. 62/193,585, filed on Jul. 17, 2015.

(51) Int. Cl. A61F 7/02; A61F 5/01; A41D 19/00

(52) U.S. Cl. CPC A61F 7/02; A41D 19/0017; A61F 5/013; A61F 2007/0036

(57) ABSTRACT

Persons find heated gloves desirable for a variety of reasons, including arthritis and cold weather. Having gloves that can be heated in a microwave provides a convenient way to supply heat. By having gloves that are fingerless, use of touch-screen devices and precise work, such as sewing and knitting, is permitted. By having gloves that offer compression, persons with conditions such as arthritis or edema may find them desirable. Additionally, compression may make limbs colder and so the ability to supply heat may be advantageous. This invention offers a set of gloves with combinations of heat, fingerlessness, and compression.

### BACKGROUND OF THE INVENTION

Gloves have been a part of daily life since ancient times. Several features are now available in gloves, but an exact combination of desirable features is not currently available.

Often gloves are manufactured without fingertip material (so-called “fingerless gloves”). This permits the gloves to warm substantially most of the hand, but leaves the fingertips free for delicate manipulation such as sewing. Modern touch-screen devices typically use capacitance to sense touch, and such devices are dramatically impaired by most gloves. Fingerless gloves also offer the advantage of permitting the use of such devices.

Often gloves are manufactured that offer compression. Such gloves are often found to be soothing for arthritis patients, for example.

Some gloves or mittens have microwave-heatable material inside them. Because of the bulk of the material, they are often formed into mittens (gloves without individual fingers) and thus hinder normal use of the hands.

In other embodiments, the heated gloves are designed to be electrically powered, which has clear disadvantages such as requiring batteries or power cords.

Generally, heated gloves apply heat to the entire hand, even though only parts of the hand are typically cold. This can result in a glove that is too warm to be comfortable.

Heated gloves also typically lack the ability to be fingerless and lack compression.

What is needed, then, is a set of gloves that offers a combination of at least some of these features. Additionally, by selectively placing microwave-heatable material next to regions of the hand where heat is desirable (and not placing it next to regions where heat is undesirable), it becomes possible to target the heat and increase comfort.

### BRIEF DESCRIPTION OF THE SEVERAL VIEWS OF THE DRAWINGS

FIG. 1 is an illustration of an embodiment of the gloves.

### DETAILED DESCRIPTION OF THE INVENTION

The following description is presented in order to enable persons of ordinary skill in the art to make and use the invention without undue experimentation. Various modifications to the disclosed embodiments will be readily apparent to persons of ordinary skill in the art, and the principles disclosed herein are applicable to other embodiments of the invention without departing from the spirit and scope of the present invention. Specifically, certain elements of the invention, such as the construction of the gloves, the addition of compression, and the location and type of microwave-heatable material, are capable of numerous modifications and variations known to persons in the art. The invention explicitly discloses that there are numerous combinations of invention elements, and all combinations are therefore disclosed herein.

The discussion below refers to a glove in the singular. It is to be understood that gloves are typically made and sold in pairs, and so the mirror-image duplicate of the described glove is also expected to be manufactured. In another embodiment the glove is reversible and so two identical gloves could be paired.

A glove 101 is constructed. The glove may have the fabric surrounding the fingertips removed (a so-called “fingerless glove”) and the fabric may be made of a material and size such that it applies compression to the hand placed inside it. (The glove is designed to be placed over a person's hand 100. Note that a person's hand has fingertips 103.)

Because the glove 101 has the fingertip material removed, the fingertips 103 are exposed. This permits the operation of touch-screens, for example, as such screens often operate on capacitance and require direct electrical conduction to the skin of the operator. This also permits use of the hands 100 to perform delicate or detailed activities such as sewing or knitting. The amount of material removed is a design choice, ranging from the very tip of the finger to down past the first knuckle.

The compression feature offers relief to some persons and for some medical ailments. In one embodiment, the compression feature is created by using elastic material that is formed to be slightly smaller than the hand 100 placed inside it. Other embodiments, such as selective placement of elastic or compressive material, are also possible.

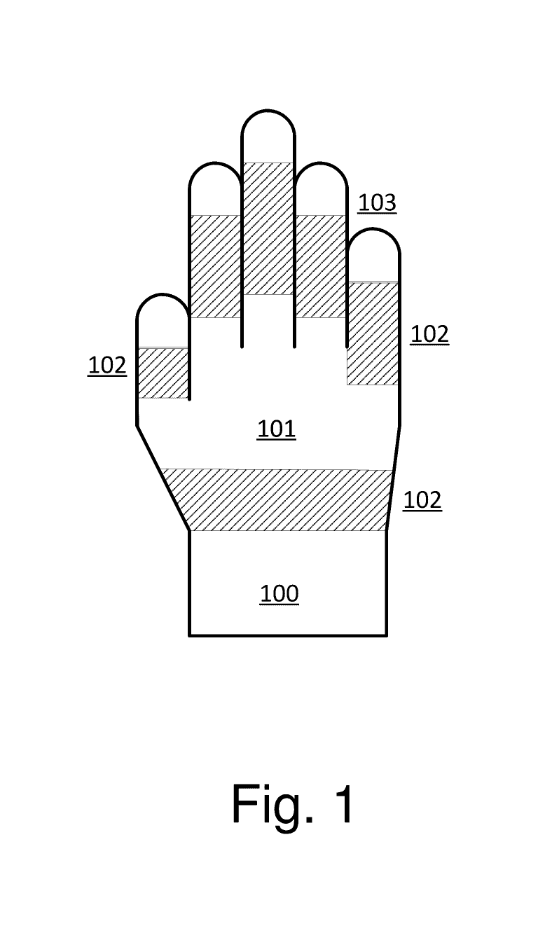

An outer layer of fabric is added in certain locations so as to retain microwave-heatable material. In one embodiment, small pouches are formed, filled with material, and attached to the outside of the glove. In another embodiment, an outer layer is attached to the glove, filled with material, and then sealed. Other embodiments, within the scope and skill of the designers of gloves and clothing, are also anticipated. Regardless of the construction method, the result is a set of regions of the glove 102, denoted in FIG. 1 with slanted lines, that can be heated in an ordinary microwave oven.

The microwave-heatable material can be any material that becomes warm in the presence of microwaves and is reusable. One example of such a material in linseeds, which also create a pleasing aroma. Dried corn and buckeyes can be used. Therapearl material, manufactured by Therapearl LLC of Columbia, Md., may also be used and has the advantage of being both heatable and capable of being cooled. The material may be in discrete units, such as seeds, or may be a gel or other semisolid material. The material may be formed into shapes corresponding to the regions of the glove where applied, or may be stuffed into pockets or pouches as needed.

In one embodiment, the microwave-heatable material surrounds the entire glove. However, this may apply too much heat or be uncomfortable. So in other embodiments, the microwave-heatable material 102 surrounds portions of the glove, as shown in FIG. 1. Such portions may correspond to areas of the hand that may benefit from the application of heat, such as the knuckles and/or wrist. Which portions to surround, and whether to surround all or part of the glove, are design decisions that can be made.

Numerous variations of the structures and feature are possible. All of these variations are within the scope and spirit of the patent.

What is claimed is:

1. A set of gloves with two or more of the following features:
Material removed from one or more fingertip areas of the said gloves so that the fingertips of a hand placed inside it are exposed
Microwave-heatable material so that the said gloves can be heated temporarily by use of a microwave oven
The said gloves apply compression to the hand inside it

2. The gloves of claim 1 such that the said microwave-heatable material does not cover the entire glove

3. The gloves of claim 2 such that the said microwave-heatable material spans one or more knuckles

4. The gloves of claim 2 such that the said microwave-heatable material span the wrist

5. A set of gloves with
Material removed from one or more fingertip areas of the said gloves so that the fingertips of a hand placed inside it are exposed
Warming means for being heatable in a microwave oven
Compressing means for applying compression to the hand inside it

6. The gloves of claim 5 such that the said warming means do not span the entire glove

7. The gloves of claim 6 such that the said warming means span at least one of the wrist or a plurality of knuckles
